SEQUENCE LISTING (8 sequences)

SEQ ID NO: 1 - RNA, 700 nt, Artificial sequence
  Base sequence of anti-sense RNA
uaccauaaaucuucacacguugcguauaguuaccucuggacauuucgaccgucugguuacgcauaucggugauugguuugguaaucguucgcgcuauaaagaauccuaggaugacugugauuaauaaugaaauagcuguaccaacaaugaauauuugauuuauauuauuuaauugguuauaaacgucauuaauuuuugauucgauauaaauauuaccaauuaccuuuuuaucgacuuuaacugggauauuauauacccagacacgguccuuaccaccgccauaaucuuuuaaaauuaaaugaucguuugauuguccuagugauagugcuuuuuggacagaacuaucauucgcuuuuugauugauuagacuacgguuagacugcuucgucgucgcaauaauaauuuggucuuuaucuauaaaacgaauuucuccaauuucuugacgguuggcauacucacuuaauaaauuuugaauaucuuuuugugcauuuacggagcccuuuucgucauauacuuuuucaauacuaauuucuaauuguuuagcguacugcguaauauucuucuuaaaauuaucaagcagcucuuuuucaagguuauuuguaaaauacagcccgauaauuugcauaccaaugauaaucaguaauacauaaacaauuacaaguuuaguaugaagggauuguaguuguuuuagccacuucau

SEQ ID NO: 2 - DNA, 700 bp, Artificial sequence
  Base sequence of expression sequence
taccataaatcttcacacgttgcgtatagttacctctggacatttcgaccgtctggttacgcatatcggtgattggtttggtaatcgttcgcgctataaagaatcctaggatgactgtgattaataatgaaatagctgtaccaacaatgaatatttgatttatattatttaattggttataaacgtcattaatttttgattcgatataaatattaccaattacctttttatcgactttaactgggatattatatacccagacacggtccttaccaccgccataatcttttaaaattaaatgatcgtttgattgtcctagtgatagtgctttttggacagaactatcattcgctttttgattgattagactacggttagactgcttcgtcgtcgcaataataatttggtctttatctataaaacgaatttctccaatttcttgacggttggcatactcacttaataaattttgaatatctttttgtgcatttacggagcccttttcgtcatatactttttcaatactaatttctaattgtttagcgtactgcgtaatattcttcttaaaattatcaagcagctctttttcaaggttatttgtaaaatacagcccgataatttgcataccaatgataatcagtaatacataaacaattacaagtttagtatgaagggattgtagttgttttagccacttcat

SEQ ID NO: 3 - DNA, 20 bp, Artificial sequence
  Sequence of primer IcaAF for PCR
gattatgtaatgtgcttgga

SEQ ID NO: 4 - DNA, 20 bp, Artificial sequence
  Sequence of primer IcaAR for PCR
actactgctgcgttaataat

SEQ ID NO: 5 - DNA, 20 bp, Artificial sequence
  Sequence of primer IcaDF for PCR
atggtcaagcccagacagag

SEQ ID NO: 6 - DNA, 23 bp, Artificial sequence
  Sequence of primer IcaDR for PCR
cgtgttttcaacatttaatgcaa

SEQ ID NO: 7 - DNA, 20 bp, Artificial sequence
  Sequence of primer yycGF for PCR
cggggcgttcaaaagacttt

SEQ ID NO: 8 - DNA, 21 bp, Artificial sequence
  Sequence of primer yycGR for PCR
tctgaacctttgaacacacgt

(19) United States
(12) Patent Application Publication
(10) Pub. No.: US 2022/0323512 A1
(43) Pub. Date: Oct. 13, 2022

(54) Methicillin-Resistant Staphylococcus Aureus Mutant Strain And Use Thereof

(71) Applicant: West China Hospital of Sichuan University, Chengdu (CN)

(72) Inventors: Shizhou Wu, Chengdu (CN); Lei Lei, Chengdu (CN); Yunjie Liu, Chengdu (CN)

(73) Assignee: West China Hospital of Sichuan University, Chengdu (CN)

(21) Appl. No.: 17/681,321

(22) Filed: Feb. 25, 2022

(30) Foreign Application Priority Data
Apr. 8, 2021 (CN) 202110381237.9

(51) Int. Cl. A61K 35/74; A61P 31/04; C12N 15/113; C12N 1/20

(52) U.S. Cl. CPC A61K 35/74; A61P 31/04; C12N 15/113; C12N 1/205; C12R 2001/445

(57) ABSTRACT

The present disclosure discloses a methicillin-resistant staphylococcus aureus mutant strain and use thereof, and belongs to the field of molecular biology and microorganisms. The methicillin-resistant staphylococcus aureus mutant strain disclosed by the present disclosure has a relatively low exopolysaccharide synthesis ability and a relatively low biofilm metabolism ability, but it is sensitive to an antibiotic cefoxitin. The mutant strain can be used for treating a related disease caused by methicillin-resistant staphylococcus aureus infection through an endogenous ecological treatment strategy. The present disclosure provides a new idea for treating the disease.

### CROSS REFERENCE TO RELATED APPLICATION(S)

This patent application claims the benefit and priority of Chinese Patent Application No. 202110381237.9, filed on Apr. 8, 2021, the disclosure of which is incorporated by reference herein in its entirety as part of the present application.

### TECHNICAL FIELD

The present disclosure belongs to the field of molecular biology and microorganisms and specifically discloses a methicillin-resistant staphylococcus aureus mutant strain and use thereof.

### BACKGROUND ART

Osteomyelitis is an infectious disease of bone cortex, bone marrow, periosteum and surrounding soft tissues caused by pathogenic microorganisms (including suppurative bacteria, mycobacteria, fungi, etc.).

The incidence of the osteomyelitis is reported to be 0.2% of the total population in developing countries. As China gradually enters an aging society, an incidence rate of diabetes rises and unexpected wounds increase, the osteomyelitis is still on the rise. Of all patients with osteomyelitis, about 37% have complications due to bone nonunion, and more 14% are at risk of amputation, thus the osteomyelitis has become one of major diseases threatening human health. Therefore, how to realize effective prevention and treatment of the osteomyelitis is an important public health problem related to national civilians.

Staphylococcus aureus is the most important pathogen for the development of the osteomyelitis, accounting for about 30% of all pathogens. It is noteworthy that currently over 50% of staphylococcus aureus has developed resistance to beta-lactam antibiotics (e.g., methicillin-resistant staphylococcus aureus (MRSA)).

### SUMMARY

The present disclosure aims to provide a methicillin-resistant staphylococcus aureus mutant strain and use thereof. The methicillin-resistant staphylococcus aureus mutant strain provided by the present disclosure has a relatively low exopolysaccharide synthesis ability and a relatively low biofilm metabolism ability, but it is sensitive to an antibiotic cefoxitin. The mutant strain can be used for treating a related disease caused by methicillin-resistant staphylococcus aureus infection through an endogenous ecological treatment strategy. The present disclosure provides a new idea for treating the disease.

The present disclosure is implemented as follows.

On the one hand, the present disclosure provides a methicillin-resistant staphylococcus aureus mutant strain. An expression of a yycG gene of the mutant strain is inhibited.

A biofilm is an organized bacterial group structure adhered to a surface of an animate or inanimate object and wrapped by extracellular macromolecules of bacteria, and has advantages of retarding penetration of antibiotics, promoting generation of drug-resistant strains and changing growth microenvironment of the bacteria. Therefore, a biofilm-forming ability of staphylococcus aureus is closely related to production of drug resistance.

Polysaccharide intercellular adhesion (PIA) is an important part of an exopolysaccharide of a biofilm of staphylococcus aureus and closely related to phenotypic changes of the biofilm. An ica gene is involved in encoding a glycosyltransferase of PIA synthesis and important for the PIA synthesis. Through a regulation of the ica gene, the PIA is anchored on a surface of the staphylococcus aureus and exerts biological functions, such that the bacteria gain adhesion and gradually form the biofilm.

Researchers of the present disclosure find that a yycG transcription system is related to the formation of the staphylococcus aureus biofilm. The system can regulate an expression of the glycosyltransferase ica gene, affect synthesis and metabolism of exopolysaccharides, change the content of biofilm matrix, and regulate sensitivity of bacteria to antibiotics.

12An exopolysaccharide synthesis ability and a biofilm formation ability of the methicillin-resistant staphylococcus aureus are reduced by inhibiting the expression of the yycG gene of the methicillin-resistant staphylococcus aureus. It is more significant that after the expression of the yycG gene is inhibited, the methicillin-resistant staphylococcus aureus is sensitive to an antibiotic cefoxitin, and has a greatly reduced drug resistance. The mutant strain has a wide range of use, more importantly can be used for treating a related disease caused by methicillin-resistant staphylococcus aureus infection through an endogenous ecological treatment strategy. Besides, the research of the present disclosure also show that the mutant strain can be used for treating the disease and has a relatively good curative effect, which is unexpected to those skilled in the art. The present disclosure can provide a new idea and strategy for treating the disease.

Preferably, in some embodiments of the present disclosure, the methicillin-resistant staphylococcus aureus mutant strain may have a deposit number of CCTCC NO:M 2020227.

The yycG gene of the mutant strain is inhibited. The methicillin-resistant staphylococcus aureus mutant strain has a relatively low exopolysaccharide synthesis ability and a relatively low biofilm metabolism ability, but it is sensitive to the antibiotic cefoxitin. The mutant strain can be used for treating the related disease caused by the methicillin-resistant staphylococcus aureus infection through the endogenous ecological treatment strategy.

A method for treating a related disease caused by the methicillin-resistant staphylococcus aureus infection through the endogenous ecological treatment strategy by using the mutant strain is as follows:

The mutant strain of the present disclosure is delivered to an infected focus to become dominant, thereby inhibiting a biofilm formation of a wild-type methicillin-resistant staphylococcus aureus to achieve a purpose of controlling an infection.

In the present disclosure, an antisense RNA expression vector can be introduced into the wild-type methicillin-resistant staphylococcus aureus to obtain a mutant strain overexpressing the antisense RNA, such that the mutant strain is dominant to the infected focus. Since a biofilm-forming ability of the mutant strain is obviously inhibited by the antisense RNA, the formation of the staphylococcus aureus biofilm in the infected focus is inhibited to achieve the purpose of controlling the infection.

On the other hand, the present disclosure provides the methicillin-resistant staphylococcus aureus mutant strain described in any one of the above in preparing a medicine for treating a related disease caused by methicillin-resistant staphylococcus aureus infection.

Preferably, in some embodiments of the present disclosure, the disease may be osteomyelitis.

The methicillin-resistant staphylococcus aureus mutant strain provided by the present disclosure is used for treating a disease including but not limited to osteomyelitis, and other diseases caused by the methicillin-resistant staphylococcus aureus mutant strain, and the methicillin-resistant staphylococcus aureus mutant strain can also be used for treatment.

On the other hand, the present disclosure provides a medicine for treating a disease, the medicine contains the methicillin-resistant staphylococcus aureus mutant strain described in any one of the above and the disease is caused by methicillin-resistant staphylococcus aureus infection.

On the other hand, the present disclosure provides a method for preparing the methicillin-resistant staphylococcus aureus mutant strain described in any one of the above and the method includes inhibiting an expression of a yycG gene of a wild-type methicillin-resistant staphylococcus aureus.

Those skilled in the art should easily understand that on the basis of the present disclosure. It is easy for those skilled in the art to think of adopting conventional gene editing methods in the art, such as but not limited to a RNA interference technology, to inhibit the expression of the yycG gene, thus the methicillin-resistant staphylococcus aureus mutant strain of the present disclosure is obtained.

Preferably, in some embodiments of the present disclosure, the RNA interference technology may be used to inhibit the expression of the yycG gene.

Preferably, in some embodiments of the present disclosure, the RNA interference technology may be used to inhibit the expression of the yycG gene: an antisense RNA expression vector capable of expressing an antisense RNA may be introduced into the wild-type methicillin-resistant staphylococcus aureus.

The antisense RNA is capable of specifically binding to a sense strand mRNA of the yycG gene to form a double-stranded RNA structure.

Preferably, in some embodiments of the present disclosure, the antisense RNA may have a base sequence as shown in SEQ ID NO.1.

A research of the present disclosure confirms that an antisense RNA shown in SEQ ID NO. 1 is capable of specifically binding to a sense strand mRNA of a yycG gene to form a double-stranded structure, accelerates the degradation of the mRNA, inhibits transcription and translation processes of the mRNA, and thus down-regulates the yycG gene. Therefore, a methicillin-resistant staphylococcus aureus shows specificity of an exopolysaccharide synthesis ability and a reduced biofilm metabolism ability, has a significantly increased sensitivity to an antibiotic cefoxitin, and thus lays a basis for using the antibiotic to treat the bacteria.

Preferably, in some embodiments of the present disclosure, an antisense RNA expression vector may have a backbone of pDL278 which contains an expression sequence for expressing the antisense RNA.

The expression sequence has a base sequence as shown in SEQ ID NO.2.

Preferably, in some embodiments of the present disclosure, the expression sequence may be located between B amHI and EcoRI restriction sites of a pDL278 plasmid vector.

On the other hand, the present disclosure provides a reagent for preparing the methicillin-resistant staphylococcus aureus mutant strain described in any one of the above and the reagent can be introduced into a wild-type methicillin-resistant staphylococcus aureus to inhibit an expression of a yycG gene.

Preferably, in some embodiments of the present disclosure, the reagent may be an antisense RNA.

Preferably, in some embodiments of the present disclosure, the antisense RNA may have a base sequence as shown in SEQ ID NO.1.

### BRIEF DESCRIPTION OF THE DRAWINGS

To describe the technical solutions in the embodiments of the present disclosure more clearly, the following briefly describes the accompanying drawings required for describing the embodiments. It should be understood that the following accompanying drawings show merely some embodiments of the present disclosure, and therefore should not be regarded as a limitation on the scope. A person of ordinary skill in the art may still derive other related drawings from these accompanying drawings without creative efforts.

FIG. 1 is a scanning electron micrograph of the strain provided in example 1 of the present disclosure;

FIG. 2 is a graph of a growth curve of the strain provided in example 1 of the present disclosure, a short line presents a wild strain and a circle presents a mutant strain;

FIG. 3 is a diagram of a staining result of the strain provided in example 1 of the present disclosure;

FIG. 4 is a schematic diagram of a comparison of a staining absorbance value of the strain provided in example 1 of the present disclosure;

FIG. 5 is a diagram of an experimental result of a protein of the strain provided in example 2 of the present disclosure, where ATCC29213 is a standard methicillin-sensitive staphylococcus aureus strain;

FIG. 6 is a comparison diagram of expression levels of glycosyltransferase-related genes of the strain provided in example 2 of the present disclosure;

FIG. 7 is a comparison diagram of an expression amount of a yycG gene provided in example 2 of the present disclosure; and

FIG. 8 is a comparison of the pathogenicity of the strains provided in example 1 of the present disclosure in mice.

### DETAILED DESCRIPTION OF THE EMBODIMENTS

Embodiments of the disclosure will be described in detail below with reference to examples, but those skilled in the art will understand that the following examples are only used to illustrate the disclosure and should not be regarded as limiting the scope of the disclosure. If no specific conditions are specified in the examples, the examples will be carried out according to conventional conditions or the conditions recommended by the manufacturer. All of the used reagents or instruments which are not specified with manufacturers are conventional commercially-available products.

In order to make the objectives, technical solutions, and advantages of the embodiments of the present disclosure clearer, the following clearly and completely describes the technical solutions in the embodiments of the present disclosure with reference to accompanying drawings in the embodiments of the present disclosure. Apparently, the described embodiments are some rather than all of the embodiments of the present disclosure. Therefore, the following detailed description of the examples of the present disclosure in the accompanying drawings is not intended to limit the protection scope of the present disclosure, but merely represent selected examples of the present disclosure. All other examples obtained by a person of ordinary skill in the art based on the examples of the present disclosure without creative efforts shall fall within the protection scope of the present disclosure.

The features and performances of the present disclosure are further described in detail below in conjunction with the examples.

### EXAMPLE 1

The present example provided a methicillin-resistant staphylococcus aureus mutant strain and the mutant strain had a deposit number of CCTCC NO:M 2020227.

The methicillin-resistant staphylococcus aureus (MRSA) mutant strain can reduce expressions of a yycG gene and a YycG protein, and had an anti-spectinomycin resistance, reduced exopolysaccharide synthesis ability and biofilm synthesis ability and increased sensitivity to an antibiotic cefoxitin.

### EXAMPLE 2

The present example provided a preparation method of the methicillin-resistant staphylococcus aureus mutant strain provided in example 1 and the preparation method included the following steps:

1.1 According to a yycG gene sequence of methicillin-resistant staphylococcus aureus, an antisense RNA sequence was designed and the antisense RNA had a base sequence as shown in SEQ ID NO.1; and a specific primer sequence (including restriction sites: BamHI/EcoRI) was designed and the antisense RNA sequence of a yycG gene was used as a template for amplification to obtain an antisense RNA expression sequence (SEQ ID No. 2) expressing the antisense RNA;

SEQ ID No. 1:
augaaguggcuaaaacaacuacaaucccuucauacuaaacuuguaauugu
uuauguauuacugauuaucauugguaugcaaauuaucgggcuguauuuua
caaauaaccuugaaaaagagcugcuugauaauuuuaagaagaauauuacg
caguacgcuaaacaauuagaaauuaguauugaaaaaguauaugacgaaaa
gggcuccguaaaugcacaaaaagauauucaaaauuuauuaagugaguaug
ccaaccgucaagaaauuggagaaauucguuuuauagauaaagaccaaauu
auuauugcgacgacgaagcagucuaaccguagucuaaucaaucaaaaagc
gaaugauaguucuguccaaaaagcacuaucacuaggacaaucaaacgauc
auuuaauuuuaaaagauuauggcggugguaaggaccgugucuggguauau
aauaucccaguuaaagucgauaaaaagguaauugguaauauuuauaucga
aucaaaaauuaaugacguuuauaaccaauuaaauaauauaaaucaaauau
ucauuguugguacagcuauuucauuauuaaucacagucauccuaggauuc
uuuauagcgcgaacgauuaccaaaccaaucaccgauaugcguaaccagac
ggucgaaauguccagagguaacuauacgcaacgugugaagauuuauggu
a.
SEQ ID No. 2:
ggatcctaccataaatcttcacacgttgcgtatagttacctctggacatt
tcgaccgtctggttacgcatatcggtgattggtttggtaatcgttcgcgc
tataaagaatcctaggatgactgtgattaataatgaaatagctgtaccaa
caatgaatatttgatttatattatttaattggttataaacgtcattaatt
tttgattcgatataaatattaccaattacctttttatcgactttaactgg
gatattatatacccagacacggtccttaccaccgccataatcttttaaaa
ttaaatgatcgtttgattgtcctagtgatagtgctttttggacagaacta
tcattcgctttttgattgattagactacggttagactgcttcgtcgtcgc
aataataatttggtctttatctataaaacgaatttctccaatttcttgac
ggttggcatactcacttaataaattttgaatatctttttgtgcatttacg
gagcccttttcgtcatatactttttcaatactaatttctaattgtttagc
gtactgcgtaatattcttcttaaaattatcaagcagctctttttcaaggt
tatttgtaaaatacagcccgataatttgcataccaatgataatcagtaat
acataaacaattacaagtttagtatgaagggattgtagttgttttagcca
cttcatggatcc.

where an underline was the restriction site;

1.2 A pDL278 plasmid vector was separately subjected to a double-enzyme digestion with BamHI and EcoRI, a linear fragment was recovered and connected with the antisense RNA expression sequence subjected to the same double-enzyme digestion obtained in the above step by a ligase to obtain a pDL278-ASyycG recombinant plasmid, that was an antisense RNA expression vector of the yycG gene of the staphylococcus aureus;

1.3 A wild-type methicillin-resistant staphylococcus aureus (isolated from an osteomyelitis focus obtained from the Department of Pathogenic Microbiology, West China Hospital, Sichuan University, Chengdu, Sichuan Province, China) was inoculated into a tryptone soybean broth (TSB) culture medium, and an anaerobic subculture overnight was conducted for later use;

1.4 2 μL of the antisense RNA expression vector pDL278-ASyycG recombinant plasmid was taken and added into 500 μL of a TSB liquid medium containing 50 μL of a wild-type methicillin-resistant staphylococcus aureus solution for transformation and the TSB liquid medium contained a competence stimulating peptide for staphylococcus aureus with a final concentration of 1m/mL;

1.5 The transformed bacterial solution in step 1.4 was subjected to an anaerobic culture at 37° C. for 150 min to obtain a transformed bacterial solution; and

1.6 The anaerobically cultured transformed bacteria solution was inoculated on a TSB plate containing spectinomycin (containing erythromycin at a final concentration of 480 μg/mL), anaerobically culture at 37° C. for 48 h, and passaged to obtain the wild-type methicillin-resistant staphylococcus aureus of example 1 (number: WL180530, hereinafter referred to as a mutant strain or WL180530, or a mutant strain WL180530);

the mutant strain was deposited in the China Center for Type Culture Collection (CCTCC) whose address was Wuhan University on Jun. 19, 2020, had a Latin name of staphylococcus aureus, was named staphylococcus aureus WS20200617 and had a deposit number of CCTCC NO:M 2020227.

It should be noted that although a wild-type methicillin-resistant staphylococcus aureus strain from a specific source is used as a basis to construct a mutant strain, in other embodiments, a wild-type methicillin-resistant from other sources can also be used to construct a mutant strain of the present disclosure, and the obtained mutant strain also has a same technical effect as the mutant strain of example 1.

### Experimental Example 1

A colony morphology, a growth performance and a biofilm formation ability of the mutant strain provided in example 1 were detected.

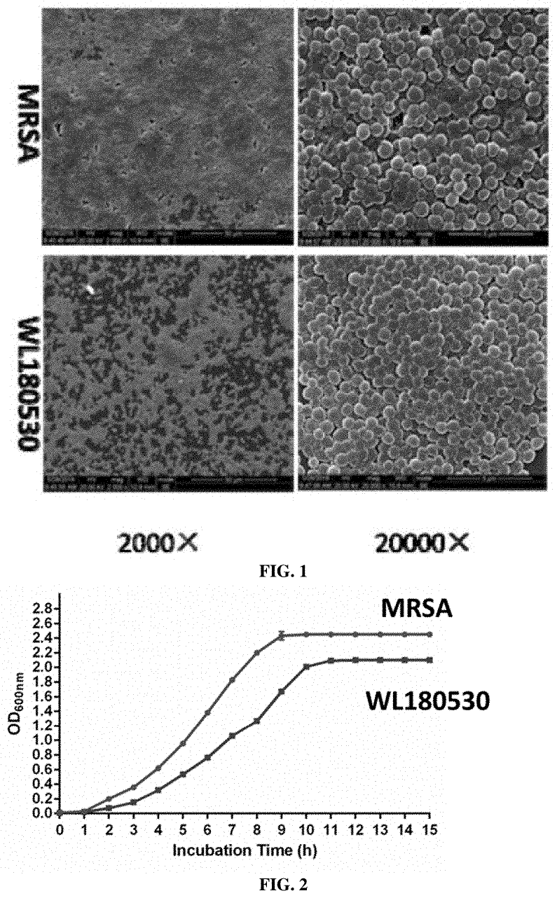

(1) Colony morphologies of the mutant strain and the wild-type methicillin-resistant staphylococcus aureus (MRSA) provided in example 1 were observed by a scanning electron microscope. The results were shown in FIG. 1.

It can be seen from the results in FIG. 1 that an extracellular matrix of a biofilm of the wild-type MRSA wrapped the bacteria, while the mutant strain (WL180530) lacked an extracellular matrix structure.

(2) Growth curves of the mutant strain WL180530 and the wild-type MRSA were determined.

The growth curves were determined as follows:

The overnight resuscitated strains were added to a fresh medium at 1:20 and anaerobically cultured at 37° C. for 24 h; and 200 μL of the bacteria were pipetted into a 96-well plate every hour, the absorbance was measured at 595 nm, and the growth curves were plotted. A result was shown in FIG. 2. It can be seen that a growth performance of the mutant strain (WL180530) was lower than that of the wild-type strain.

(3) A film formation ability of the mutant strain WL180530 and the wild-type MRSA was determined. An experiment was conducted as follows: the strain was diluted into a bacterial suspension of 5×10⁵ CFU/mL and 200 μL of the bacterial suspension was added to a 96-well plate and anaerobically cultured at 37° C. for 24 h; the floating bacteria and a supernatant thereof were aspirated and washed with a PBS buffer twice; methanol was used to fix the biofilm of the strain, the biofilm was dried at room temperature and stained with 100 μL of 0.1% (W/V) crystal violet for 5 min; the stained biofilm was washed with deionized water, 200 μL of 95% ethanol was added for reabsorption, and the absorbance value was measured at 595 nm.

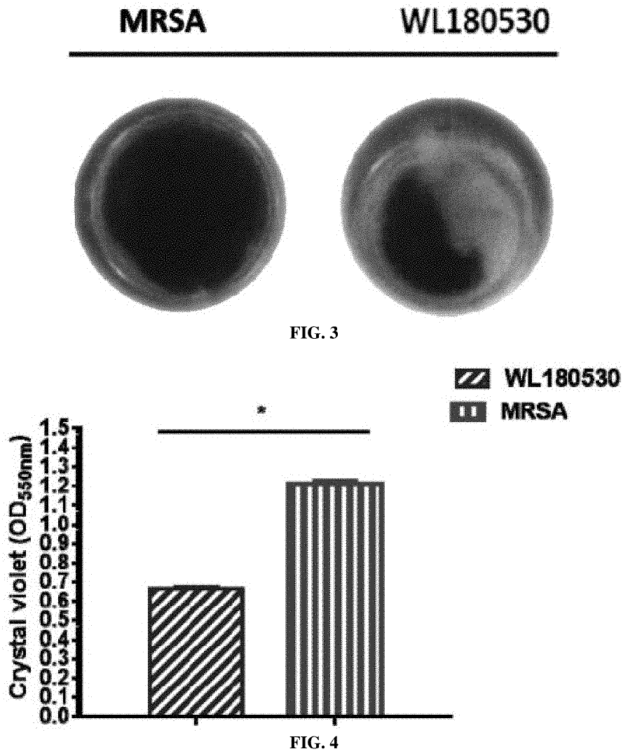

A staining result and an absorbance determination result were shown in FIG. 3 and FIG. 4. As can be seen from FIG. 3, the mutant strain WL180530 had a staining ability significantly lower than that of the wild-type MRSA, indicating the mutant strain WL180530 had a reduced biofilm formation ability. As can be seen from FIG. 4, the mutant strain WL180530 had a significantly lower absorbance value than the wild-type MRSA, which further indicated that the mutant strain WL180530 had the reduced biofilm formation ability.

### Experimental Example 2

A yycG gene expression of the mutant strain provided in example 1 was detected.

(1) A YycG protein of the mutant strain WL180530 and a wild-type methicillin-resistant staphylococcus aureus was extracted and subjected to an SDS-PAGE test and a western blot test. The protein was extracted as follows: the overnight resuscitated strains were added to 10 mL of a fresh medium at 1:20 and anaerobically cultured at 37° C. to a mid-logarithmic growth phase, centrifugation was conducted at 4° C., the bacterial cells were collected, dissolved in a lysozyme solution and subjected to wall breaking in an ultrasonic manner, centrifugation was conducted and a supernatant total protein was collected.

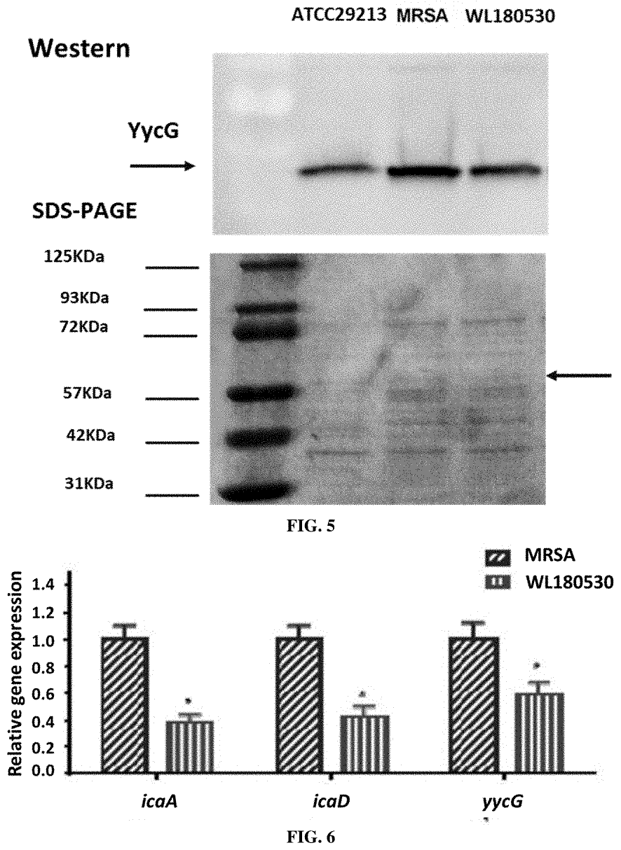

The results were shown in FIG. 5. It can be seen from FIG. 5 that the YycG protein band of the mutant strain WL180530 was narrow, indicating that a protein expression was decreased.

(2) Expression levels of the yycG gene and glycosyltransferase-related genes icaA/D of the mutant strain WL180530 and the wild-type methicillin-resistant staphylococcus aureus were detected by using an SYBR Green I chimeric fluorescence method. A real-time PCR amplification was conducted. An ABI PRISM 7300 was used to carry out a PCR amplification procedure according to a two-step method. Primer sequences were as shown in Table 1.

TABLE 1
Fluorescence quantitative PCR primers

| Primer name | No. | Sequence |
|---|---|---|
| IcaA-F | SEQ ID No. 3 | gattatgtaatgtgcttgga |
| IcaA-R | SEQ ID No. 4 | actactgctgcgttaataat |
| IcaD-F | SEQ ID No. 5 | atggtcaagcccagacagag |
| IcaD-R | SEQ ID No. 6 | cgtgttttcaacatttaatgcaa |
| yycG-F | SEQ ID No. 7 | cggggcgttcaaaagacttt |
| yycG-R | SEQ ID No. 8 | tctgaacctttgaacacacgt |

IcaA-F SEQ ID No. 3 gattatgtaatgtgcttgga IcaA-R SEQ ID No. 4 actactgctgcgttaataat IcaD-F SEQ ID No. 5 atggtcaagcccagacagag IcaD-R SEQ ID No. 6 cgtgttttcaacatttaatgcaa yycG-F SEQ ID No. 7 cggggcgttcaaaagacttt yycG-R SEQ ID No. 8 tctgaacctttgaacacacgt

It can be seen from FIG. 6 that the expression levels of the glycosyltransferase-related genes such as icaA and icaD in the mutant strain WL180530 decreased and the expression of the yycG gene in the mutant strain WL180530 decreased significantly.

### Experimental Example 3

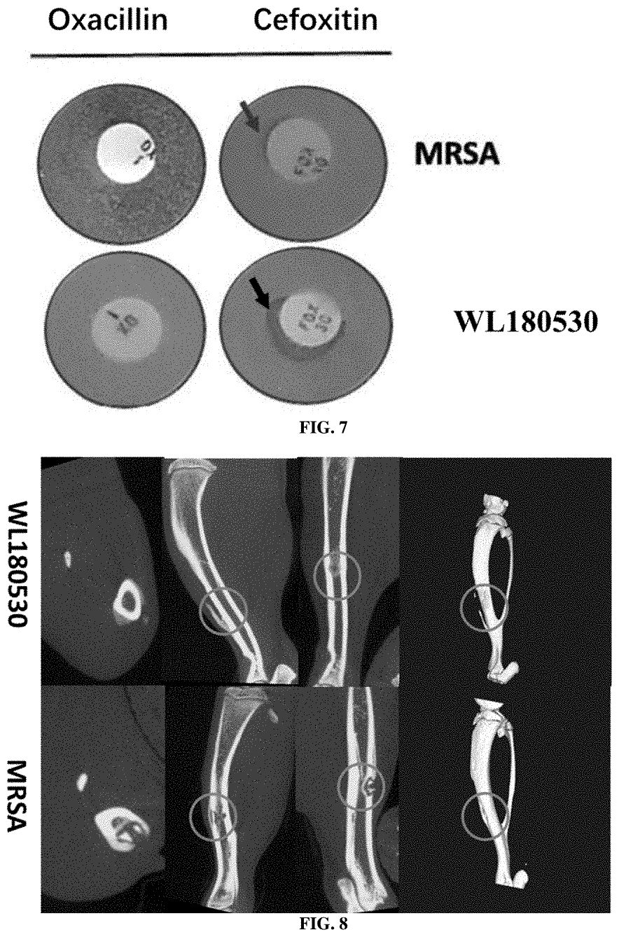

A sensitivity of a mutant strain to antibiotics was detected by using a bacteriostatic circle test. It can be seen from FIG. 7 that the mutant strain WL180530 had increased sensitivity to the antibiotic cefoxitin.

### Experimental Example 4

Pathogenicity of a mutant strain was compared in a rat tibial osteomyelitis model: a female SD rat was anesthetized by intraperitoneal injection of 10% chloral hydrate at a dose of 200 g/mL and fixed in a supine position after the successful anesthesia. A right tibia of the rat was subjected to skin preparation and routinely disinfected. A surgical drape was spread. An incision about 1 cm long was cut in an anterior inner side of a ⅓ upper segment of a shank, a tibial cortex was exposed, a Kirschner wire with a diameter of 0.1 cm was used for a drilling preparation and a bone marrow cavity was exposed. After the exposure, 40 μL of a methicillin-resistant staphylococcus aureus (MRSA) standard strain (an MRSA group) and 40 μL of a LASyycG MRSA mutant strain (a WL180530 group) in a mid-logarithmic growth phase were injected into the tibial bone marrow cavity of the rats in different groups. After the injection, the bone marrow cavity was sealed with bone wax and the skin incision was closed layer by layer. After four weeks of observation, the rats were subjected to a tibia Micro-CT detection under a general anesthesia. It can be seen from FIG. 8 that the rats in the mutant strain WL180530 group had significantly reduced bone defect, indicating that the mutant strain WL180530 had the weakened pathogenicity.

The described examples are merely some rather than all of the examples of the present disclosure. The detailed description examples of the present disclosure are not intended to limit the protection scope of the present disclosure, but merely represent selected examples of the present disclosure. All other examples obtained by a person of ordinary skill in the art based on the examples of the present disclosure without creative efforts shall fall within the protection scope of the present disclosure.

SEQUENCE LISTING

<110> West China Hospital, Sichuan University
<120> METHICILLIN-RESISTANT SMETHICILLIN-RESISTANT STAPHYLOCOCCUS
AUREUS MUTANT STRAIN AND USE THEREOF APHYLOCOCCUS AUREUS MUTANT
STRAIN AND USE THEREOF
<130> GWP202107512
<160> 8
<170> Patentin version 3.5
<210> 1
<211> 700
<212> RNA
<213> Artificial sequence
<220>
<223> Base sequence of anti-sense RNA
<400> 1

| | |
|---|---|
| uaccauaaau cuucacacgu ugcguauagu uaccucugga cauuucgacc gucugguuac | 60 |
| gcauaucggu gauugguuug guaaucguuc gcgcuauaaa gaauccuagg augacuguga | 120 |
| uuaauaauga aauagcugua ccaacaauga auauuugauu uauauuauuu aauugguuau | 180 |
| aaacgucauu aauuuuugau ucgauauaaa uauuaccaau uaccuuuuua ucgacuuuaa | 240 |
| cugggauauu auauacccag acacgguccu uaccaccgcc auaaucuuuu aaaauuaaau | 300 |
| gaucguuuga uuguccuagu gauagugcuu uuuggacaga acuaucauuc gcuuuuugau | 360 |
| ugauuagacu acgguuagac ugcuucgucg ucgcaauaau aauuuggucu uuaucuauaa | 420 |
| aacgaauuuc uccaauuucu ugacgguugg cauacucacu uaauaaauuu ugaauaucuu | 480 |
| uuugugcauu uacggagccc uuuucgucau auacuuuuuc aauacuaauu ucuaauuguu | 540 |
| uagcguacug cguaauauuc uucuuaaaau uaucaagcag cucuuuuuca agguuauuug | 600 |
| uaaaauacag cccgauaauu ugcauaccaa ugauaaucag uaauacauaa acaauuacaa | 660 |
| guuuaguaug aagggauugu aguuguuuua gccacuucau | 700 |

<210> 2
<211> 700
<212> DNA
<213> Artificial sequence
<220>
<223> Base sequence of expression sequence
<400> 2

| | |
|---|---|
| taccataaat cttcacacgt tgcgtatagt tacctctgga catttcgacc gtctggttac | 60 |
| gcatatcggt gattggtttg gtaatcgttc gcgctataaa gaatcctagg atgactgtga | 120 |
| ttaataatga aatagctgta ccaacaatga atatttgatt tatattattt aattggttat | 180 |
| aaacgtcatt aatttttgat tcgatataaa tattaccaat taccttttta tcgactttaa | 240 |
| ctgggatatt atatacccag acacggtcct taccaccgcc ataatctttt aaaattaaat | 300 |
| gatcgtttga ttgtcctagt gatagtgctt tttggacaga actatcattc gctttttgat | 360 |
| tgattagact acggttagac tgcttcgtcg tcgcaataat aatttggtct ttatctataa | 420 |
| aacgaatttc tccaatttct tgacggttgg catactcact taataaattt tgaatatctt | 480 |
| tttgtgcatt tacggagccc ttttcgtcat atactttttc aatactaatt tctaattgtt | 540 |
| tagcgtactg cgtaatattc ttcttaaaat tatcaagcag ctctttttca aggttatttg | 600 |
| taaaatacag cccgataatt tgcataccaa tgataatcag taatacataa acaattacaa | 660 |
| gtttagtatg aagggattgt agttgtttta gccacttcat | 700 |

<210> 3
<211> 20
<212> DNA
<213> Artificial sequence
<220>
<223> Sequence of primer IcaAF for PCR
<400> 3

| | |
|---|---|
| gattatgtaa tgtgcttgga | 20 |

<210> 4
<211> 20
<212> DNA
<213> Artificial sequence
<220>
<223> Sequence of primer IcaAR for PCR
<400> 4

| | |
|---|---|
| actactgctg cgttaataat | 20 |

<210> 5
<211> 20
<212> DNA
<213> Artificial sequence
<220>
<223> Sequence of primer IcaDF for PCR
<400> 5

| | |
|---|---|
| atggtcaagc ccagacagag | 20 |

<210> 6
<211> 23
<212> DNA
<213> Artificial sequence
<220>
<223> Sequence of primer IcaDR for PCR
<400> 6

| | |
|---|---|
| cgtgttttca acatttaatg caa | 23 |

<210> 7
<211> 20
<212> DNA
<213> Artificial sequence
<220>
<223> Sequence of primer yycGF for PCR
<400> 7

| | |
|---|---|
| cggggcgttc aaaagacttt | 20 |

<210> 8
<211> 21
<212> DNA
<213> Artificial sequence
<220>
<223> Sequence of primer yycGR for PCR
<400> 8

| | |
|---|---|
| tctgaacctt tgaacacacg t | 21 |

1-10. (canceled)

11. A methicillin-resistant staphylococcus aureus mutant strain, wherein an expression of a yycG gene of the mutant strain is inhibited.

12. The methicillin-resistant staphylococcus aureus mutant strain according to claim 11, wherein the methicillin-resistant staphylococcus aureus mutant strain has a deposit number of CCTCC NO:M 2020227.

13. A method for preparing the methicillin-resistant staphylococcus aureus mutant strain according to claim 11, comprising inhibiting an expression of a yycG gene of a wild-type methicillin-resistant staphylococcus aureus.

14. A method for preparing the methicillin-resistant staphylococcus aureus mutant strain according to claim 12, comprising inhibiting an expression of a yycG gene of a wild-type methicillin-resistant staphylococcus aureus.

15. The method according to claim 13, wherein a RNA interference technology is used to inhibit the expression of the yycG gene.

16. The method according to claim 14, wherein a RNA interference technology is used to inhibit the expression of the yycG gene.

17. The method according to claim 15, wherein the RNA interference technology is used to inhibit the expression of the yycG gene: an antisense RNA expression vector capable of expressing an antisense RNA is introduced into the wild-type methicillin-resistant staphylococcus aureus;
the antisense RNA is capable of specifically binding to a sense strand mRNA of the yycG gene to form a double-stranded RNA structure; and
preferably, the antisense RNA has a base sequence as shown in SEQ ID NO.1.

18. The method according to claim 16, wherein the RNA interference technology is used to inhibit the expression of the yycG gene: an antisense RNA expression vector capable of expressing an antisense RNA is introduced into the wild-type methicillin-resistant staphylococcus aureus;
the antisense RNA is capable of specifically binding to a sense strand mRNA of the yycG gene to form a double-stranded RNA structure; and
preferably, the antisense RNA has a base sequence as shown in SEQ ID NO.1.

19. The method according to claim 17, wherein the antisense RNA expression vector has a backbone of pDL278 which contains an expression sequence for expressing the antisense RNA; and
the expression sequence has a base sequence as shown in SEQ ID NO.2.

20. The method according to claim 18, wherein the antisense RNA expression vector has a backbone of pDL278 which contains an expression sequence for expressing the antisense RNA; and
the expression sequence has a base sequence as shown in SEQ ID NO.2.

21. A reagent for preparing the methicillin-resistant staphylococcus aureus mutant strain according to claim 11, wherein the reagent is capable of being introduced into wild-type methicillin-resistant staphylococcus aureus and inhibiting the expression of the yycG gene;
preferably, the reagent may be an antisense RNA or an expression vector thereof; and
preferably, the antisense RNA has a base sequence as shown in SEQ ID NO.1.

22. The reagent for preparing the methicillin-resistant staphylococcus aureus mutant strain according to claim 21, wherein the methicillin-resistant staphylococcus aureus mutant strain has a deposit number of CCTCC NO:M 2020227.
